(19) United States
(12) Patent Application Publication
(10) Pub. No.: US 2014/0025027 A1
(43) Pub. Date: Jan. 23, 2014

(54) FUNCTIONAL FABRICS, PROTECTIVE GARMENTS MADE THEREFROM, AND METHODS OF MAKING

(71) Applicants: Enqin Eunice Png, Providence, RI (US); Julie M. Sygiel, Providence, RI (US); Daniel M. Wyner, North Scituate, RI (US)

(72) Inventors: Enqin Eunice Png, Providence, RI (US); Julie M. Sygiel, Providence, RI (US); Daniel M. Wyner, North Scituate, RI (US)

(73) Assignee: Dear Kate, Inc, Providence, RI (US)

(21) Appl. No.: 13/876,105

(22) PCT Filed: Sep. 27, 2011
(86) PCT No.: PCT/US11/53459

§ 371 Date: Oct. 9, 2013

Related U.S. Application Data
(60) Provisional application No. 61/386,980, filed on Sep. 27, 2010.

(51) Int. Cl. A61F 13/66

(52) U.S. Cl. CPC A61F 13/665
USPC 604/371; 604/385.15

(57) ABSTRACT

Garments comprising functional fabrics and methods of making such garments are disclosed. In certain embodiments, an undergarment is disclosed comprising at least one moisture-absorbent layer of fabric having a first body-contacting side and a second side, and at least one moisture-repellent layer of fabric having a first side disposed adjacent to the second side of the moisture-absorbent layer, and a second side; and a leg cuff comprising a portion of the moisture-repellent layer, a seam and an elongate elastic member.

### TECHNICAL FIELD

The present disclosure is related to functional fabrics, protective garments made therefrom, particularly protective undergarments, and methods of making the foregoing.

### BACKGROUND

Many varieties of feminine sanitary products have been commercialized, and are designed to absorb bodily discharges. For instance, sanitary napkins and tampons exist in many designs and iterations with slightly varying functions to absorb menstrual fluid. However, many products are not fully adequate, allowing menstrual discharges to leak onto women's inner and outer apparel, often leaving stubborn stains on the apparel, especially if allowed to dry before washing. Some women immediately may throw away stained undergarments, but many attempt to remove the stains using a variety of cleansers and techniques. Without thorough cleansing, stains may become permanent, which may be a source of embarrassment if others see the stains. When a leak extends to a woman's outerwear, it may require the involvement of a cleaning service, which is even more embarrassing.

Although leaks may occur at any time, women tend to experience them more often in the following two instances: on the first day of the menstrual cycle, when not yet wearing appropriate sanitary products, and when lying down, usually during sleep. However, various vaginal discharges with the potential to stain garments occur throughout the month.

Some undergarments have been designed to solve the issue of menstrual leaks and staining. However, such products are either not adequately protective and have no barrier properties (offering only absorbent characteristics), or alternatively, they are constructed with barrier fabrics that are aesthetically unpleasing and uncomfortable.

Diapers and adult incontinence products are generally absorbent on the inside, but because they need to absorb very large amounts of liquid, they use thick pads where aesthetics are completely different from the constructions of the present concept. Because the fluid levels are highly varied and quite large in these products, they also use complete film barriers that are generally not air permeable.

White these garments have some functionality, none of them are suitable for use as an undergarment for daily use,

There is a need for functional fabric and garments made therefrom that absorb fluid, prevent the transmission of fluid to outerwear, do not permanently stain, and additionally are sufficiently attractive and comfortable to wear on a daily basis in place of non-protective undergarments.

### SUMMARY OF THE INVENTION

The present disclosure is directed to washable undergarments and portions of such undergarments comprising functional fabrics, that is, fabrics having desirable functional characteristics, such as the moisture absorbency, moisture repellence, and the like, in preferred embodiments, the functional fabrics and the garments made thereof retain their desirable functional characteristics after multiple washings. The present disclosure is directed, in one embodiment, to a washable undergarment, comprising at least one moisture-absorbent layer of fabric comprising a body-contacting surface, and an absorbent capacity of at least about 300 g/m²; and at least one moisture-repellent layer disposed adjacent to the at least one moisture-absorbent layer and comprising an outer surface disposed opposite the body-contacting surface. In some embodiments, the undergarment comprises a lining or a gusset having 1-4 moisture-absorbent layers and a shell comprising 1-2 moisture-repellent layers. In certain embodiments, the garment comprises at least one layer of fabric having an absorbent capacity of about 347 to about 806 g/m²for a single fabric layer. In embodiments having multiple fabric layers, the total absorbency of the combined fabric layers is about 806 g/m² to about 1178 g/m². In embodiments having multiple fabric layers, the combined fabric layers have an absorbent capacity of about 38 to about 50 ml of fluid.

The present disclosure is directed, in another embodiment, to a garment portion, comprising at least one moisture-absorbent layer comprising a body-contacting surface, and an absorbent capacity of at least about 300 g/m²; and at least one moisture-repellent layer disposed adjacent to the at least one moisture-absorbent layer and comprising an outer surface disposed opposite the body-contacting surface. In certain embodiments, the garment portion is a gusset or a lining. In certain embodiments, the gusset comprises 1-4 moisture-absorbent layers and 1-2 moisture-repellent layers. Typically, the gusset comprises at least one layer of fabric having an absorbent capacity of about 347 to about 806 g/m² for a single fabric layer. In embodiments having multiple fabric layers, the total absorbency of the combined fabric layers is about 806 g/m² to about 1178 g/m². In embodiments having multiple fabric layers, the combined fabric layers have an absorbent capacity of about 38 to about 50 ml of fluid.

In another embodiment, the present disclosure is directed to a garment and portions thereof comprising at least one moisture-absorbent layer comprising a body-contacting surface; and at least one moisture-repellent layer disposed adjacent to the at least one moisture-absorbent layer and comprising an outer surface disposed opposite the body-contacting surface; wherein the garment portion comprises a combined weight of less than about 20 oz/yd².

In another embodiment, the present disclosure is directed to a garment and portions thereof comprising at least one moisture-absorbent layer comprising a body-contacting surface; and at least one moisture-repellent layer disposed adjacent to the at least one moisture-absorbent layer and comprising an outer surface disposed opposite the body-contacting surface; wherein the garment portion comprises a combined absorbent capacity of at least about 40 ml. In certain preferred embodiments, the total water absorbency of the combined fabric layers is about 806 g/m² m to about 1178 g/². In certain embodiments, the combined absorbent capacity is about 37 ml to about 50 ml.

Any one of the foregoing garments and portions thereof may be transmissive to air and/or transmissive to moisture vapor. One or both surfaces of the layers can comprise a napped, sueded or texturized surface, adapted to absorb fluid and to wick the absorbed fluid through to the adjacent layer.

The at least one moisture-absorbent layer and the at least one moisture-repellent layer can be polymeric material or a microfiber material, and combinations thereof. The polymeric material can comprise polyolefins, polyamides, and combinations thereof.

In certain embodiments, the at least one moisture-repellent layer can comprise a water repellant finish, which may be a film having a thickness of less than about 10 microns (μm). In some embodiments, the film may be driven into the fabric by heat and pressure. In other embodiments, the at least one moisture-repellent layer is a meltblown material such as a stretchable polymer such as polyurethane or co-polyether-ester, or a microfiber layer. In further embodiments, the at least one moisture-repellent layer can comprise a fabric which has been coated or saturated with a silicone or polyurethane or other elastic water repellent polymer. In preferred embodiments, a moisture-repellent layer is a knitted fabric that has been treated with a moisture-repellent polymer emulsion.

In some embodiments, the at least one moisture-absorbent layer and at least one moisture-repellent layer may be bonded together, and the bonding may be at an edge of each layer.

Another aspect of the disclosure is an undergarment comprising one of the foregoing garment portions, which is a gusset portion of the undergarment. The gusset can comprise a front edge, a side, a back edge and opposing side edges, and the front and back edges are attached to a front and a back of the undergarment at a front seam and a back seam. One or more of the front and back seams comprise an inverted triangular shape, and/or one or more of the front and back seams extend to an upper edge of the undergarment. In some embodiments, one or more of the front and back seams can comprise a hydrophobic coating. In other embodiments, one or more of the opposing edges can comprise a hydrophobic coating. In yet other embodiments, one or more of the front and back seams and one or more of the opposing edges can comprise a hydrophobic coating.

In further embodiments, the garment portion is a lining portion of the undergarment that is approximately the same size and shape as a moisture-repellent shell garment portion that forms the outer surface of the garment. In certain embodiments, the undergarment can further include waistband portions, leg cuffs and decorative trim.

### BRIEF DESCRIPTION OF THE DRAWINGS

Referring now to the figures, which are exemplary embodiments, and wherein like elements are numbered alike:

FIG. 1A, FIG. 1B, FIG. 1C, and FIG. 1D show front, back, interior and exterior views, respectively, of one exemplary garment according to the present disclosure;

FIG. 2A, FIG. 2B, FIG. 2C, and FIG. 2D show front, back, interior and exterior views, respectively, of another exemplary garment according to the present disclosure;

FIG. 3A, FIG. 3B, FIG. 3C, and FIG. 3D show front, back, interior and exterior views, respectively, of another exemplary garment according to the present disclosure;

FIG. 4A, FIG. 4B, FIG. 4C, and FIG. 4D show front, back, interior and exterior views, respectively, of another exemplary garment according to the present disclosure;

FIG. 4E, FIG. 4F, FIG. 4G, and FIG. 4H show front, back, interior and exterior views, respectively, of another exemplary garment according to the present disclosure;

FIG. 5 shows a cross-sectional view of one exemplary multi-layer fabric according to the present disclosure;

FIG. 6 shows a cross-sectional view of another exemplary multi-layer fabric according to the present disclosure;

FIG. 7 shows a cross-sectional view of an exemplary dual-layer fabric according to the present disclosure;

FIG. 8 shows Table A, which lists the characteristics of various hydrophobic and hydrophilic fabrics;

FIG. 9 shows Table B, which summarizes the qualitative test results of panties of working Examples 1-8 that were constructed using various combinations of fabrics; and

FIG. 10 shows Table C, which summarizes the absorbent capacity of selected panties of working Examples 1-8.

### DETAILED DESCRIPTION

The present disclosure is directed to protective garments that provide leak resistance, fluid absorbance and fluid barrier characteristics. In some instances, the garments also may provide stain resistance and/or stain-releasing characteristics. The protective garments are stretchy and breathable, have a non-film-like drape, and an attractive look and feel. Exemplary protective garments according to the present disclosure are protective undergarments, particularly women's protective undergarments, which can be made aesthetically attractive, similar to non-protective women's undergarments, thereby offering women the ability to wear the protective undergarments without discomfort or embarrassment.

The undergarments can comprise at least one layer of a fabric that is hydrophilic, or treated to be hydrophilic, disposed adjacent to at least one layer of fabric that is hydrophobic or treated to by hydrophobic. Alternatively, the undergarments can comprise a single layer of fabric with a first surface that is hydrophilic or treated to be hydrophilic, and second surface that is hydrophobic or treated to be hydrophobic, opposite the hydrophilic surface. In certain embodiments, the multilayered assembly of functional fabric that includes at least one layer of a fabric that is moisture-absorbent or treated to be moisture-absorbent and at least one layer of a fabric that is moisture-repellent or treated to be moisture-repellent is located in the region of a panty normally occupied by a gusset, and is referred to as a “gusset.” In other embodiments, the multilayered assembly of functional fabric is more extensive in size, comprising most of the fabric in the garment. In such embodiments, “lining” is used to refer to the layers of moisture-absorbent fabric and optional layer of moisture-repellent fabric and “shell” is used to refer to the outer layer of moisture-repellent fabric. In the disclosure of working examples, below, the component used in each layer is identified.

In preferred embodiments, the present garments are constructed to absorb and/or contain the volume of fluid from a woman's menstrual cycle, which may be in the range of 5-30 milliliters and varies, of course, depending on many factors.

The present garment construction does not require a film or coating, which tends to be uncomfortable or undesirable for the reasons noted above. The foregoing characteristics are accomplished with the use of unique functional fabrics and garment constructions. The present undergarments provide effective leak resistance against and/or act as a fluid barrier against Hood spills, while remaining breathable and stretchable.

In certain embodiments, the fabrics may be stretchable knit fabrics, which provide leak resistance and/or act as a fluid barrier, in the absence of a film or coating. The use of such fabrics for leak resistance and/or as a fluid barrier is unique, with or without the use of elastane (e.g., LYCRA®) in the fabric.

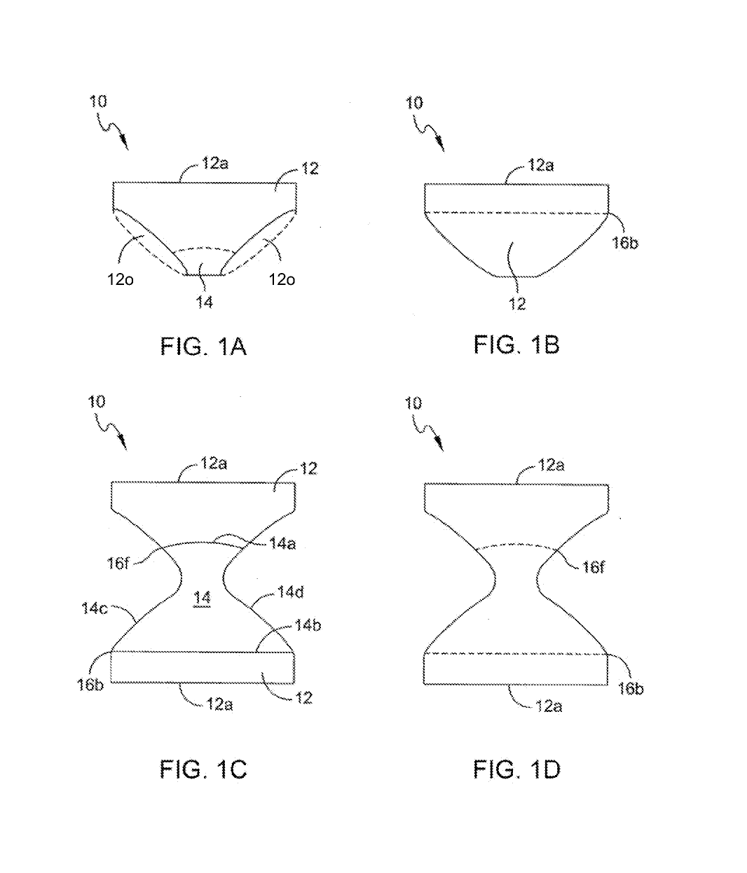

FIG. 1A-FIG. 1D show an exemplary embodiment of an undergarment 10 according to the present disclosure, which will be referred to hereinafter for ease of illustration as a panty 10. Panty 10 comprises a body portion 12 and a gusset region 14. Body portion 12 comprises an upper edge 12a defining a waistband, and leg openings 12o. Gusset region 14 comprises front and back edges 14a, 14b, and opposing side edges 14c, 14d. Gusset region 14 is attached to the body portion 12 at the front and back edges 14a, 14b, defining a front seam 16f and a back seam 16b. Gusset region 14 comprises a functional material 100 according to the present disclosure, as will be described in further detail below.

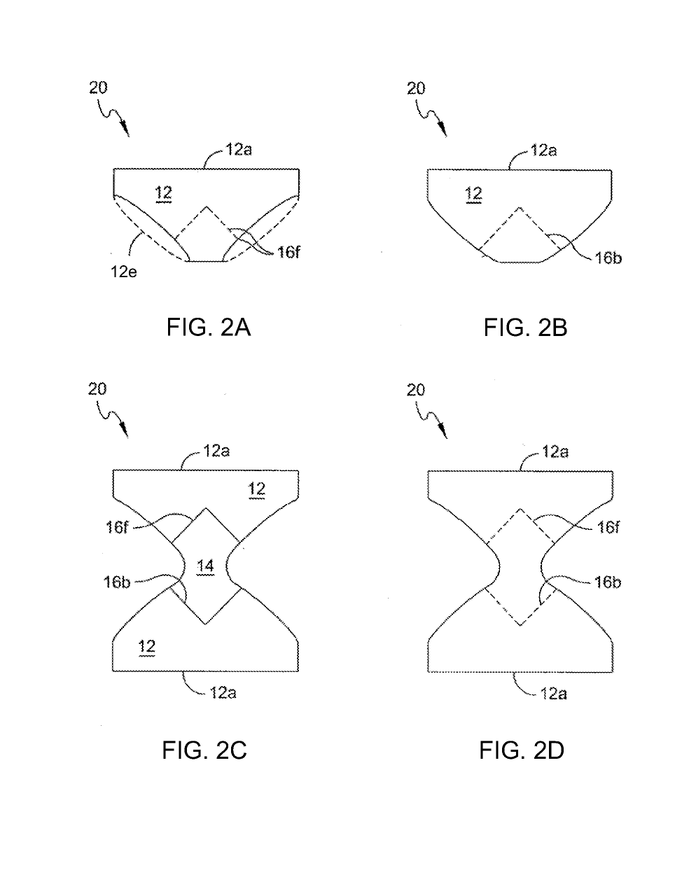

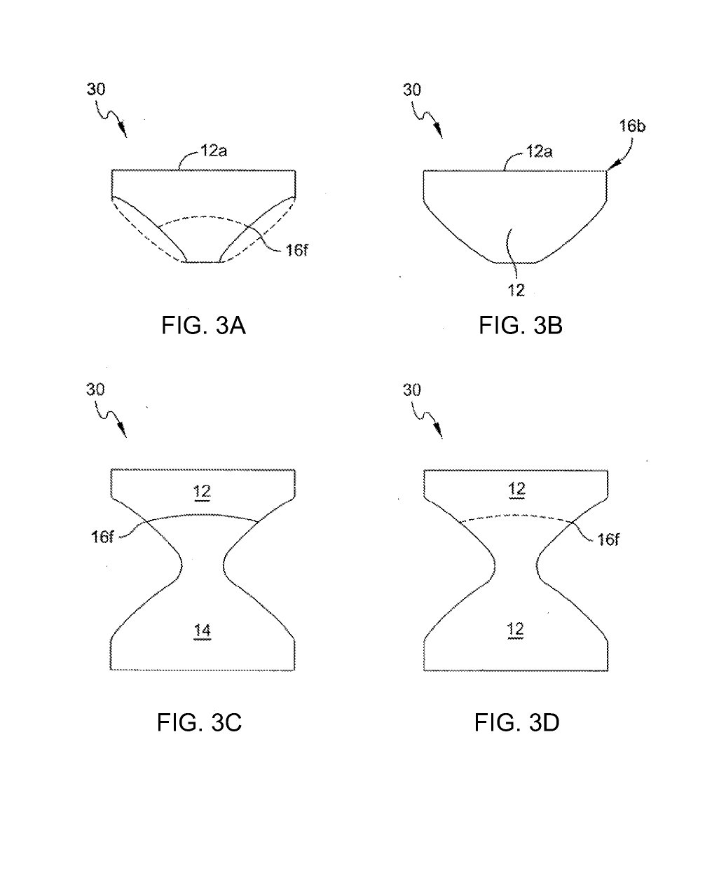

Optionally, the shape and position of the front and back seams 16f, 16b may be varied relative to the upper edge 12a, to accommodate stains that are sometimes found in the front and/or back areas of the underwear, especially at the edge of the front and back of sanitary napkins. For example, FIG. 2A-FIG. 2D show another exemplary embodiment of an undergarment 20 in which the front and back seams 16f, 16b form the shape of the edges of a vertex of an inverted triangle. Such a shape assists in preventing leaks, spotting, and stains on the front and back of the underwear. Also optionally, gusset 14 can extend to the upper edge 12a of the panty, as shown in FIG. 3A-FIG. 3D show in which the gusset 14 extends up to and is co-terminus with the upper edge 12a of the panty. An edge 12e of the body 12 is indicated.

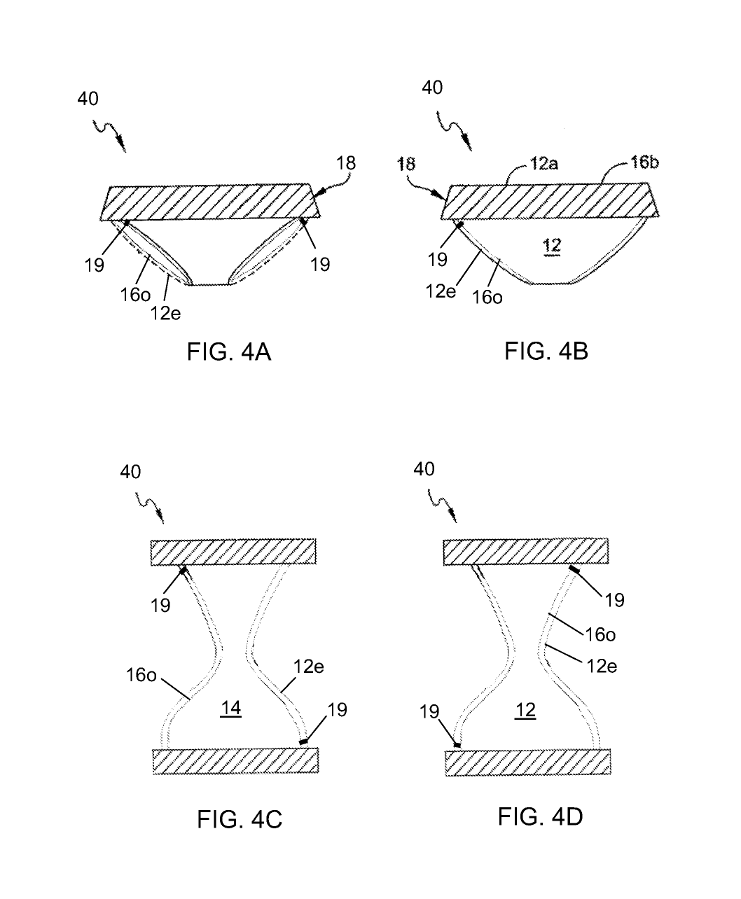

Alternatively or in addition to, the panty can comprise one piece of fabric continuing to the aforementioned front seam, as shown in FIG. 4A-FIG. 4H.

FIG. 4A-FIG. 4D show another exemplary embodiment of an undergarment. 40, such as a bikini panty or thong panty, according to the present disclosure. To maximize the underwear's function and aesthetics, seams, like those that would be created by underwear designs shown above, may be avoided by constructing the panty so that the entire front to back may be made of the aforementioned functional fabrics. Such a construction involves attaching the gusset or lining 14 at the front and back edges 14a, 14b to a band 18 of fabric that has the same or a different composition from that of the main body or shell 12 of the panty. FIG. 4E-FIG. 4H show a further exemplary embodiment of an undergarment 40a, such as a hipster panty or cheeky panty, according to the present disclosure. As shown, the band 18 of fabric may comprise lace, silk, chiffon, cotton, or any fabric that may or may not be treated to have functional properties, but more importantly, prevents visible panty lines around the waist area. In preferred embodiments, the functional fabric lining of the undergarment may or may not be limited to the gusset region between the leg openings.

In certain embodiments, the edges of the body 12 and the gusset or lining 14 joined in a leg opening seam 16o that is offset from the edge 12e of the body 12. An elongate elastic member, such as an elastic tape, may be layered between the body 12 and the gusset 14, lying between the leg opening seam 16o and the edge 12e of the body at the leg opening. In certain preferred embodiments, the elastic can be secured using bartack stitching 19. In other embodiments, the elongate elastic member can be elastic decorative trim, such as elastic lace. When the leg opening seam 16o, the elongate elastic member and at least one moisture-repellent layer of fabric participate in the formation of a leg cuff in the finished undergarment.

In any and all of the foregoing embodiments, the gusset or lining 14 of the undergarment may be composed of one or more separate pieces of fabric that are not bound together. This design allows for the wings of a sanitary pad to be inserted between the separate layers of fabric in a discreet and comfortable way.

One embodiment is designed similar to regular non-functional underwear. However, the entire garment may be made of functional fabric (excluding decorative embellishments), with features as described above, or with no seams such that the entire underwear, excluding the band 18, is made of one single piece of fabric. By constructing the entire tower section of the underwear with functional fabrics, the underwear can protect the wearer of this garment from unwanted stains and leaks without compromising style. However, the upper area of the garment does not need to have such special functionalities, and thus may be made out of any fabric to lower the cost of the garment. This is true unless the fabric on the lower part of the garment has immense wicking capabilities that result in the transfer of fluids to the top band of the garment. Then, the band may be treated with a hydrophobic finish. Even so, this may decrease the overall cost of the garment as compared to the entire underwear constructed with the invented combination of fabrics, and this may increase the aesthetics and functionalities of the underwear.

Optionally, a seam binding may be sewn over the seam edges of the layers the gusset region, to prevent the edges of the gusset or lining from rolling. “Seam edge,” as used herein, means the cut edge of the fabric that is spaced apart from and adjacent to a seam. The binding may be sewn over the seam edge so that it is exposed on one or both of the inside and the outside of the leg openings in the gusset region.

Alternatively, the layers in the gusset region may be attached using a sewing technique, such that the seam attaching the gusset layers together is disposed on the inside of the gusset region and is spaced apart from the leg opening, so that both the inside and the outside of the gusset region does not have any exposed or visible seam threads. With this construction, any wicking along the seams will be drawn to the interior of the gusset, between the layers, rather than to the outside of the panties and/or to outer clothing, which might otherwise result in a stain. Optionally, a binding may be sewn over the seam edge in the gusset region, so that the binding is disposed between the layers, which minimizes the edges of the gusset from rolling during use.

In certain embodiments, such as those showing in FIG. 4A-FIG. 4D and FIG. 4E-FIG. 4H, in which the gusset or lining 14 and the body or shell 12 are substantially co-extensive, the seam attaching the gusset layers together is disposed on the inside of the gusset region and is spaced apart from the leg opening, so that both the inside and the outside of the gusset region does not have any exposed or visible seam threads, as described above, and encircles the leg opening. In other embodiments, the seam threads inside and the outside of the gusset region are visible. As shown in FIG. 4A-FIG. 4H, the seam 16o is spaced apart front the edge 12e of the body 12. When assembled, the seam 16o and edge 12e lie on either side of an elongate elastic member, such as elastic tape (not shown) that is secured at least in part by bartack stitching 19 form a complex of structures that define a leg cuff, indicated as 12o in FIG. 1A. In certain preferred embodiments, the seam stitching and bartack stitching comprise non-wicking thread. In preferred embodiments, the elongate elastic member has been treated to be moisture-repellent. In other embodiments, the elongate elastic member can be elastic decorative trim, such as elastic lace.

Any and all of the materials, fabrics, layers and techniques described above may be combined, or may be used individually.

The present disclosure also provides fabrics that may be used in the foregoing and other garments. “Fabric,” as used herein, refers to a single or multiple layers of fabrics. The present fabric and garment constructions provide unique advantages of stain resistance, fluid retention, fluid absorbency, and garment aesthetics (look, feel, softness and stretch) that are comparable to those of standard (non-protective) undergarments. The garments are not intended to replace feminine hygiene products such as sanitary napkins and tampons, but to complement such products during a woman's menstruation.

In certain embodiments, a single layer of fabric suitably has a weight in the range of about 3.7 oz/yd² to about 7.2 oz/yd². In other embodiments, a single layer of fabric suitably has a weight in the range of about 7 oz/yd² up to about 14.6 oz/yd². In certain embodiments that comprise two-five layers of fabric, the combined fabric weight is suitably in the range of about 9.5 oz/yd² to about 24.9 oz/yl².

In preferred embodiments, the fabric comprises a fabric layer or layers with a total absorbent capacity of at least about 300 g/m² as determined using the Eulie Dip Test, more preferably, at least about 800 g/m². In particularly preferred embodiments. the fabric comprises a fabric layer or layers with a total absorbent capacity of at least about 1000 g/m². In preferred embodiments, the total absorbent capacity of the gusset or lining is about 806 g/m² to about 1178 g/m². In certain embodiments, the absorbent capacity of an undergarment is about 38 to about 50 ml as measured by the Eulie capacity test.

It is desirable for the inner layer to absorb liquids fairly rapidly to avoid spills, and thus the inner layer should have an absorbency percentage of at least 10% as determined in ASTM D4772. It is important that the fabric actually absorb liquid into the fibers of the fabric rather than simply hold liquid in the space between fibers.

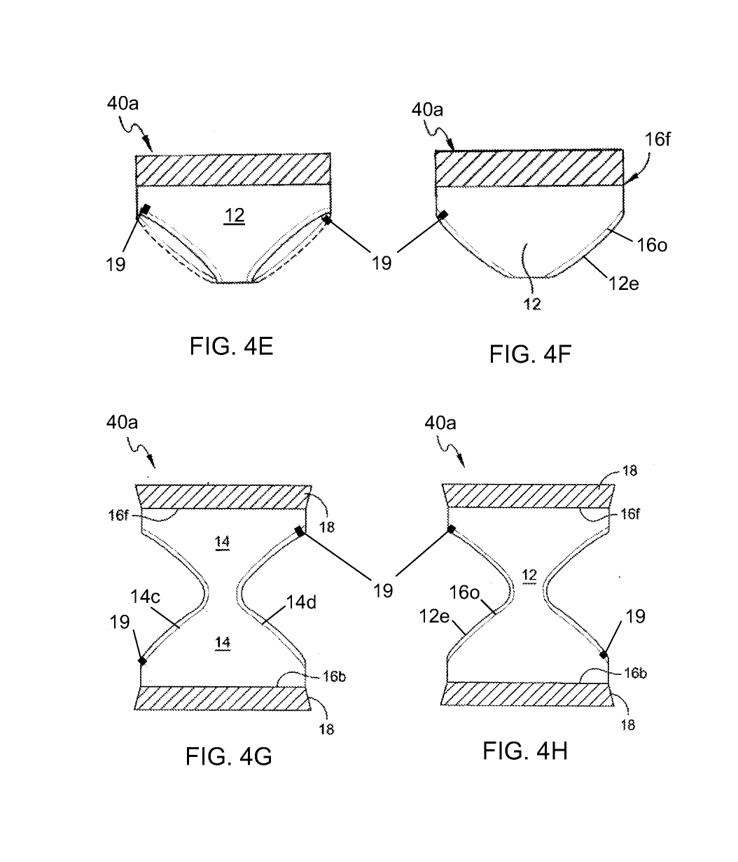

FIG. 5 shows one exemplary multi-layer fabric section 50 that can be used in any garment or garment portion, including any of the foregoing embodiments. Fabric section 50 comprises four layers: a first, or inner, layer 100 having a first body contacting surface and a second surface; a second, moisture-absorbent layer 200 having a first surface disposed adjacent to the second surface of inner layer 100, and a second surface; a third, fluid-repellent or fluid-proof barrier layer 300 having a first surface disposed adjacent to the second surface of the moisture-absorbent layer 200, and a second surface; and, an optional fabric layer 400 that has a first surface and a second surface that may be included for, among other things, aesthetic reasons. When present, the first surface of layer 400 is disposed adjacent to the second surface of the barrier layer 300.

Inner layer 100 may be any material that is capable of allowing the transmission of fluid to the absorbing layer 200, and thereby remains relatively dry even when fluids penetrate its surface. The dryness of inner layer 100 may be achieved through several methods. For example, when fluid is released on a point source of the inner surface (body-contacting surface) of inner layer 100, the fabric can distribute the fluid within the layer 200 by wicking the liquid across a greater area. Alternatively, layer 100 may be non-absorbent, such that the majority of the fluid is transferred to layer 200, allowing layer 100 to remain free of fluid and, consequently, stains. The stains in layer 200 then not apparent from the perspective of the wearer.

Suitable materials for the body contacting layer 100 include, but are not limited to, fabrics made from the following inherently stain-resisting fibers: polyolefin, polyamide, polyester, and combinations thereof For ease of discussion, the term “stain-releasing,” will be used herein to mean both stain-resistant materials and stain-releasing materials, including those that have been treated to be stain-resistant or stain-releasing. Alternatively, the layer 100 can comprise any suitable fabric that has been treated with a stain releasing or resisting finish (such as the Darlington finishes listed in Table A). Layer 100 can also comprise an inherently stain-releasing material such as microfiber or a microfiber blend comprising different materials that can be treated to have even greater stain-releasing capabilities.

Alternatively, or in addition to the foregoing, layer 100 may have a relatively dark color that can assist in masking stains.

The moisture-absorbent layer 200 can comprise any material capable of absorbing fluid, and of releasing the absorbed fluid under certain conditions (for example, during a laundry cycle). The moisture-absorbent layer can comprise an absorbency of greater than about 300 grams/per square meter (“gm/m²”); more particularly greater than about 800 gm/m², and more particularly still greater than about 1054 gm/m². In preferred embodiments, the total absorbent capacity of the gusset or lining is about 806 g/m² to about 1178 g/m².

Suitable materials for the moisture-absorbent layer 200 include, but are not limited to, woven or nonwoven microfiber or polymer knits; fabrics formed using hydrophilic fibers, absorbent or superabsorbent foams, fibers or powders.

Alternatively, any knit, absorbent knit, woven, nonwoven or polymeric material that has reservoir properties due to air gaps or voids can be used as the moisture-absorbent layer 200. Examples of such materials include, but are not limited to, double needlebar knit fabrics, foams, nonwovens, and the like.

In some embodiments, the moisture-absorbent layer 200 can comprise a knitted fabric that has been treated to have hydrophilic properties. Additionally, the yarn from with the knitted fabric is made can be treated prior to knitting to be hydrophilic. In some embodiments, the yarn and the knitted fabric can both be treated to be hydrophilic.

Alternatively, it is possible that the moisture-absorbent layer could be removable and possibly disposable, reducing or eliminating the requirement for stain resistance. Such as disposable layer may comprise, for example, a thin absorbent or superabsorbent foam, fabric, nonwoven or composite.

Alternatively, the moisture-absorbent layer 200 can be covered with an aesthetic non-staining inner layer 100 that can mask some level of staining, which would then remain invisible to the consumer,

The barrier layer 300 can comprise any material or combinations of materials that prevent or minimize the transmission of fluid through the barrier layer, and that do not adversely affect the feel and/or hand of the garment.

While many materials may be used in combination for the barrier layer 300, we have devised a unique test for measuring elongation. We have found that the use of woven or non-woven fabrics having elongation test results comparable to the Darlington fabrics listed in Table A to be effective.

By having an moisture-absorbent layer 200 capable of absorbing the specified quantity of fluids, the hydrostatic pressure resistance of the barrier layer 300 can be moderate, which allows more latitude for creating desirable aesthetics.

Some suitable materials for the barrier layer 300 include, but are not limited to, a fabric laminated to a polymer film. The polymer film can comprise a thickness of less than or equal to about 15 microns, more particularly less than or equal to about 10 microns, and more particularly still less than or equal to about 5 microns. Suitable materials for the polymer film include, but are not limited to, copolyether-esters, thermoplastic elastomers (TPEs) such as HYTREL®, nylons, and polyolefins. Other soft elastic TPEs could be used if they have sufficient softness and pliability to be used in an undergarment. The polymer film may be laminated in a 2-ply or 3-ply configuration, or may be free-hanging and sewn between other layers, such as between an absorbent layer 300 and an aesthetic layer 400. One suitable polymer material is a highly moisture transmitting monolithic polyurethane film sold by Omniflex Greenfield, Mass., under the name TX1540.

Other suitable materials for the barrier layer 300 include, but are not limited to, a 2-ply laminate polymer film protected by another layer that is not bonded to the film, as this arrangement allows for a softer feel in the finished garment. The film/composite material may optionally be subjected to a hydrophobic (water-repellent) treatment to enhance the barrier properties without negatively impacting the hand of the material. In still another embodiment, the barrier film or composite can be stretched or pressed with heat and/or pressure into the surrounding layers to enhance feel. These stretch or heat and pressure treatments also have the advantage of creating small fissures that enhance breathability.

Other suitable materials for barrier layer 300 can include a hydrophobic fabric that may be inherently hydrophobic, or which may be treated to make it hydrophobic. Suitable fabrics for treating with such hydrophobic coatings include, but are not limited to, relatively tight knitted, nonwoven or woven fabrics. Suitable hydrophobic materials for treating the fabric include, but are not limited to, polymers such as silicone, polyurethane and combinations thereof. In many embodiments, it is desirable to use elastic polymers for this purpose, such as LYCRA® and blends thereof In certain embodiments, it may be desirable for the hydrophobic treated knitted, nonwoven or woven material to contain microfibers, as this creates a more tortuous path for any fluid to penetrate.

If the material used to form the barrier layer is nonwoven, the use of a meltblown nonwoven material may be desirable, since it also provides a tortuous path for fluids. Multiple nonwoven, knitted or woven materials may also be combined in any number of layers. Several or all layers may be treated with hydrophobic treatment or made of inherently hydrophobic material(s). Embodiments using such treated (non-film) materials may provide greater breathability than may be achieved with a film barrier. One exemplary barrier layer is an elastic meltblown nonwoven material made from a copolyether-ester polymer similar to the product previously sold by Kimberly-Clark under the brand name DEMIQUE®.

In other embodiments, the barrier layer 300 can suitably comprise a microporous polymer film. Suitable microporous polymer films include, but are not limited to, urethane films, polytetrafluoroethytene (PTFE) films, polyolefin films, and combinations thereof. One suitable microporous urethane film is sold by Porvair PLC, Norfolk, UK. Other suitable microporous PIPE films are available under the product name GORE-TEX® (W. L. Gore & Associates, Inc., Newark, Del.). Such microporous films can be used in any combination of layers, either laminated or unlaminated, and can be treated with a hydrophobic water repellent treatment, or filled with a substance such as oil, to keep the pores from becoming contaminated.

Various materials can be used as the optional aesthetic layer 400. Examples of suitable materials for layer 400 include, but are not limited to, lace, silk, chiffon, cotton, polyester, nylon, LYCRA®, and the like, and blends and combinations thereof. If desired, the fabric can be colored, printed, etc., and may be treated to have any of the functional properties described herein.

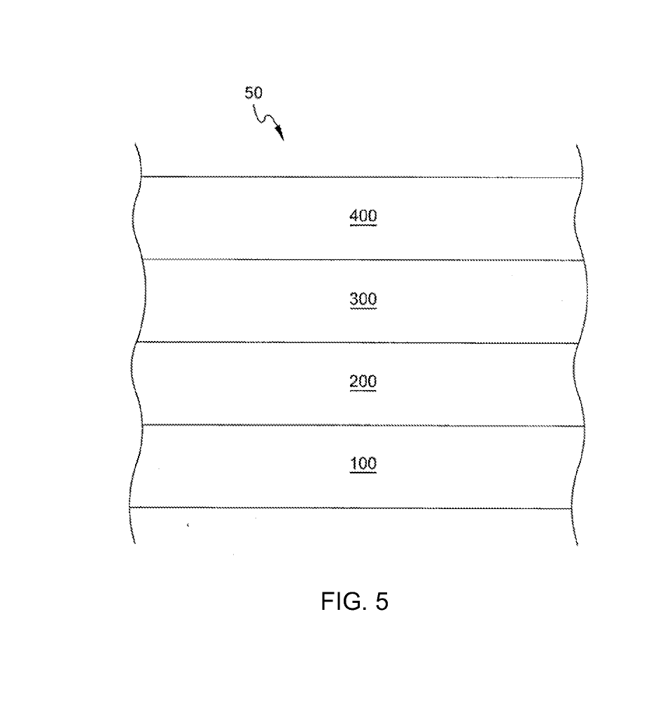

FIG. 6 shows another exemplary multi-layer fabric section 60 that can be used in the present garments, comprising three layers: a first, body contacting and absorbent layer 500; a second, fluid-resistant or fluid-proof barrier layer 300 disposed adjacent to the absorbent layer 500, and an optional fabric layer 400. Each layer has a first surface and second surface, which are disposed adjacent to the surface(s) of the neighboring layers(s) as described with reference to FIG. 5. Suitable materials for layers 300 and 400 are the same as those described above in FIG. 5.

Suitable materials for layer 500 include any material that is both stain-resistant and that is capable of absorbing fluid and, under certain conditions, releasing the fluid. Examples of suitable materials for layer 500 include materials discussed above with respect to layer 200 of FIG. 5. The body-contacting surface of layer 500 can be treated with a material that imparts stain resistance. An example of such a treatment would be the Darlington treatments listed in Table A, however any similar treatment would be suitable.

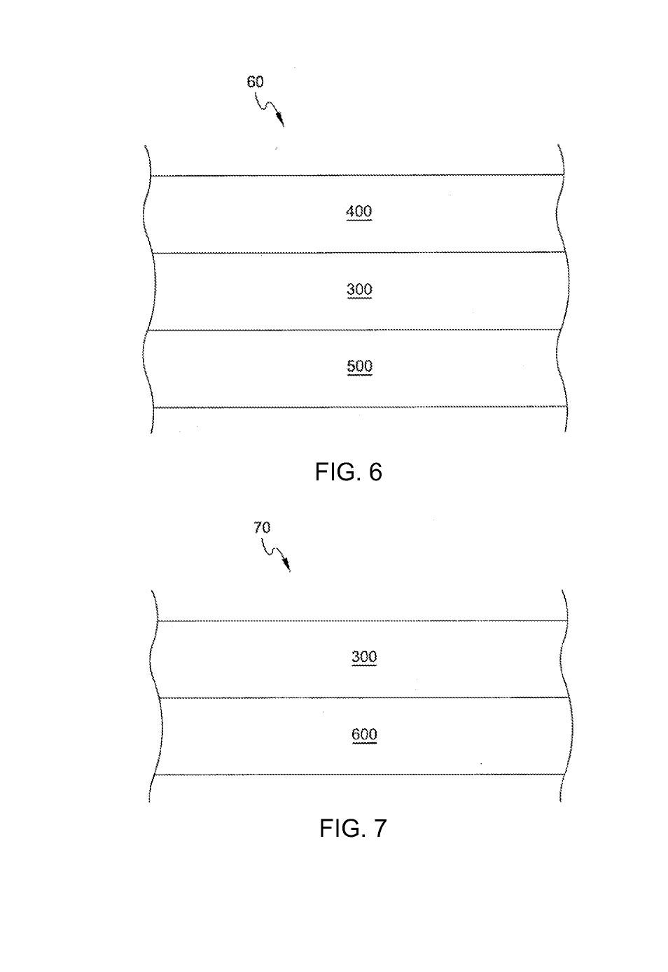

FIG. 7 shows another exemplary, dual-layer, fabric section 70 that can be used in the present garments, comprising a first, body contacting layer 600 and an optional fabric layer 400. Each layer has a first surface and second surface, which are disposed adjacent to the surface(s) of the neighboring layers(s) as described with reference to FIG. 5. The functional features of such embodiments can be combined in a single layer 600 of fabric that combines fluid absorption and barrier characteristics and, in certain embodiments, stain-releasing characteristics. Such a fabric may be constructed of one or more fibers with one or more of the foregoing characteristics, combining the characteristics of the different fibers to achieve the desired undergarment properties. In some cases, it may be desirable for a single fabric layer to have the different surfaces of the fabric faced with different types of fibers, each type of fiber having different characteristics. In certain such embodiments, one type of fiber is hydrophobic and absorbent, and one type of fiber is hydrophilic.

In some embodiments, layer 600 can comprise a single layer of fabric having a first surface that is stain resistant (inherently, or as a result of being treated with a stain resistant treatment, as described above) and moisture-absorbent, and a second surface that is hydrophobic. The first surface can be capable of absorbing an amount of fluid between about 0.1 milliliters (“ml”) and about 50 ml of fluid within as little as 95 cm² up to the entire surface area of the undergarment, and more particularly between about 3 ml and about 15 ml fluid. The second, opposite surface of the layer can be treated to be hydrophobic (as described above) and to prevent or minimize the transmission of the absorbed fluids. The second surface may be treated with a conventional durable water-repellent (DWR) treatment (such as ZEPEL® or other treatments) and/or it may be saturated or impregnated with a hydrophobic polymer (such as silicone or urethane). The hydrophobic polymer fills the gaps in the fabric, and may be applied by dip and squeeze, knife over roll, spray, gravure, or other methods. The fabric used may be, for example, a knit and with the stretch characteristics described above. The fabric may also include the use of microfiber.

Improved performance may be obtained by bonding the seams together without stitching using tape or adhesive seams, or using sealants over the stitched seams. Suitable materials include elastic polymer sealants and adhesives. Seams can be sealed with seam tape such as Worthen Industries (Nashua, N.H.) Tape 375-4, using a Pfaff seam sealing machine to eliminate sewing the edges of the gusset together or to the body of the garment. Non-wicking thread may also be used to minimize or eliminate blood from wicking along the thread in stitched seams.

In any and all of the embodiments disclosed herein, two or more of layers 100 through and including 600 may be attached at least partially to each other using a variety of techniques, in order to reduce the number of layers in the garments. For example, the layers may be laminated to form a single, composite layer, or they may be attached by sewing at various attachment points, so that the separate layers remain detached in between the seams.

Also, any or all of the materials and/or material surfaces in the undergarments can comprise an active agent, such as an antimicrobial or antifungal material. Example of suitable active agents include, but are not limited to, ionic silver, copper, zinc, nanoparticles thereof, and combinations thereof (which act as a natural antimicrobials). The use of nanoparticles does not compromise the aesthetics of the garments, which is important for undergarments. The active agents can be added to any layer of fabric or film or in fact into the adhesive if one is used for bonding. The addition of phase-change microspheres may also be used to add a temperature-regulating feature.

The foregoing fabrics and composites facilitate the construction of many garments, particularly aesthetically pleasing and protective women's undergarments. The undergarment designs may be slightly different from non-protective undergarments to aid leak and stain prevention, while simultaneously being aesthetically pleasing. Accordingly, the foregoing materials and methods of construction may be applied to styles that characterize regular non-protective undergarments, such as briefs, thongs, boy shorts, and the like. Existing “period” underwear is either designed such that the protective gusset is of regular size or the protective gusset extends to the back waistband in a fashion that is unappealing. The present concept provides designs are functional preventing leaks and stains but are also aesthetically pleasing.

The functional fabrics discussed above have many other uses including but not limited to regular women's and men's apparel, men's functional apparel, industrial fabrics, sporting apparel, and protective apparel. The garment and fabric constructions described herein are designed for use in protective intimate apparel to absorb and/or contain bodily discharges and to resist staining and/or release staining when laundered, while not negatively impacting the look, feel and breathability for use in intimate apparel and other clothing. The garments and construction methods described herein may be used for a variety of garments including but not limited to underwear, bras, bathing suits, and outerwear.

While one use for this unique combination of materials is for protective panties during menstruation, the present concept may also be used for underwear and outerwear preventing leaks and stains during mild incontinence episodes, pregnancy, post-partum, menopause, and post-menopause. The capabilities may be used separately or combined in panties, bras, outer clothing, bathing suits, and the like. The potential wicking capabilities may be used specifically in, but not limited to, outerwear and sleepwear for menopausal women. In addition, pregnant and post-partum women often experience unexpected lactation, causing uncomfortable and embarrassing stains and/or teaks on bras and/or outer garments. The use of this concept for construction of nursing bras can alleviate this inconvenience. Pregnant and post-partum women also experience heavy vaginal bleeding during pregnancy and after giving birth, creating a strong need for stain releasing and leak proof products. In addition, pre-teen girls often experience anxiety about menstruating for the first time. The present materials and construction techniques provide garments that would allow them to feel at ease knowing they are protected from potential leaks or stains.

### WORKING EXAMPLES

### Fabric Testing

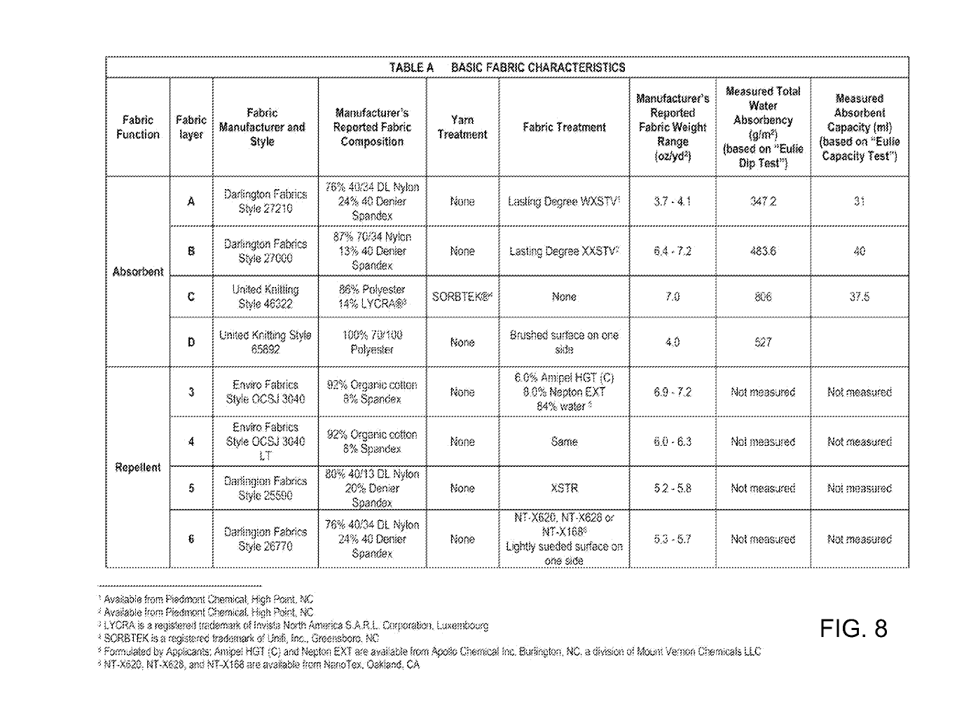

Two categories of fabrics were tested for use in the present garments: moisture-absorbent (hydrophilic) fabrics and moisture-repellent (hydrophobic) fabrics. Table A (FIG. 8) summarizes the fabric type, yarn type and treatment type, if any, as well as the manufacturer's reported fabric weight, the total water absorbency (as determined by the “Eulie Dip Test,” discussed below), and the measured absorbent capacity (as determined by the “Eulie Capacity Test, discussed below). Throughout the Tables, the moisture-absorbent layer fabrics are referred to by a letter designation A, B or C, and the moisture-repellent layer fabrics are referred to by a numeric designation 3, 4 or 5.

### Eulie Dip Test

As noted above, the total water absorbency of each fabric was tested using the Eulie Dip Test, which involves measuring, cutting and weighing a five inch by five inch (5″×5″) dry piece of fabric. The fabric piece was then fully submerged in water for 15 seconds, after which it was removed from the water while holding only the upper corners. The excess water was allowed to drain from the fabric piece for twenty-five (25) seconds, after which the fabric piece was weighed a second time to determine the wet fabric weight. The total water absorbency was calculated by subtracting the dry fabric weight from the wet fabric weight, as shown in Table A.

### Eulie Capacity Test

Also as noted above, the absorbent capacity of each fabric was tested using the Eulie Capacity Test, which involves which involves measuring, cutting and weighing a five inch by five inch (5″×5″) dry piece of fabric. The fabric section was disposed on an angled surface of five (5) degrees, and the tip of a titration tube was disposed one (1) centimeter (cm) above the fabric surface, four (4) cm from the upper edge of the fabric section, centered on both sides of the fabric section. Fluid (water containing green food coloring) was dispensed from the titration tube at a flow rate of approximately 20 milliliters/minute. Fluid flow was discontinued when water ran off the edge of the fabric section, or when water reached all four corners of the fabric section. The absorbent capacity listed in Table A represents the total volume of water dispensed from the titration tube.

### Functional Trials

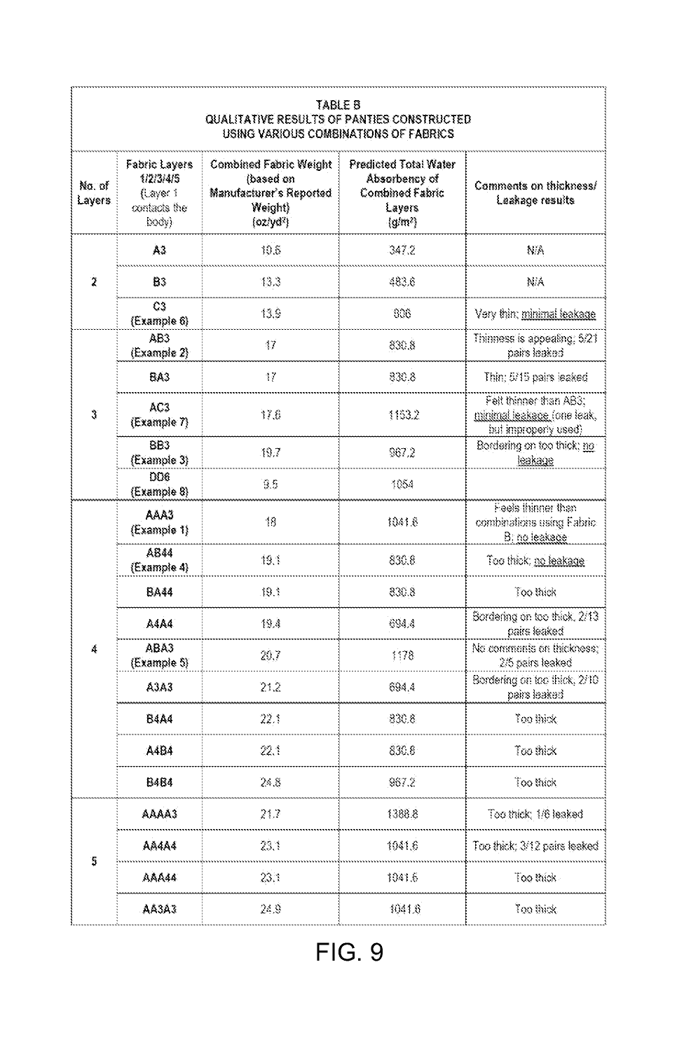

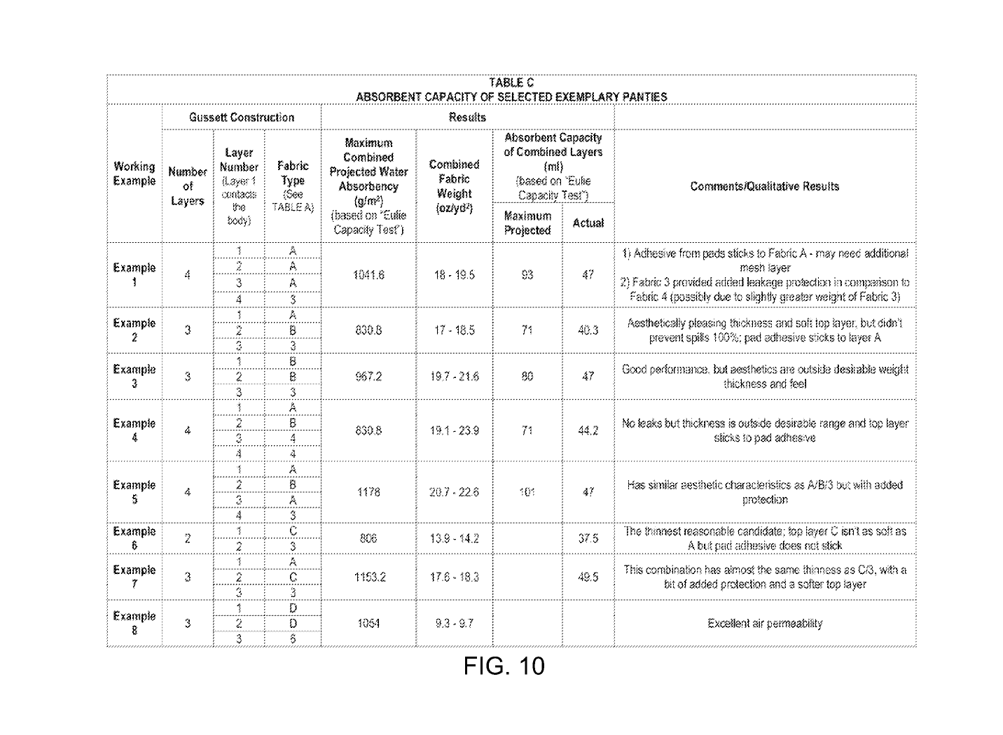

The fabrics listed in Table A were used to construct panties for Functional Trials by women during their menstrual cycle. The panties were constructed by forming test gussets of the fabrics listed in Table A of FIG. 8, in various combinations. The test gussets were then stitched over the gusset region of various commercially available panties (e.g., HANES®, etc.). The details of the specific combinations and the test results are provided in Table B of FIG. 9, and Table C of FIG. 10,

Eight working example gussets were constructed for testing. The fabrics are identified using the system of Table A, where moisture-absorbent fabrics are identified by the letters A, B, C, and D, and moisture-repellent fabrics are identified by the numbers 3, 4, 5, and 6. For example, the two-layer gusset of working Example 6 was constructed with a moisture-absorbent inner layer of United Knitting style 46322 (fabric C) contacting the wearer's body and a moisture-repellent layer of Enviro Fabrics style OCSJ 3040 treated with a water repellent composition comprising 6.0% Amipel HOT (C), 8.0% Nepton EXT (fabric 3). In addition, four three-layer gussets (Example 2, AB3, Example 3, BB3, Example 7, AC3, and Example 8, DD6) and three four-layer gussets (Example 1, AAA3, Example 4, AB44, and Example 5ABA3) were tested. In each combination, the wearer's skin was in contact with a moisture-absorbent layer.

The panties were forwarded to volunteer testers. The testers wore the panties while menstruating, and recorded the amount of time that the panties were worn and the amount of time between wearing and washing the panties. After washing, a record of any remaining stains was recorded by the testers, along with qualitative remarks about the comfort and effectiveness of the panties.

There was an aesthetic trade-off when the gusset became too bulky or thick due to multiple layers. Therefore, it is desirable to provide the maximum amount of absorbent capacity per thickness or weight of the absorbent layers. Some of the best performing composites from the functional trial included 3 layers of thin moisture-absorbent layer “A” (Example 1), but a single moisture-absorbent layer C (Example 6) also appears to perform well.

Fabric A appeared to absorb liquids initially faster than Fabric B, resulting in fewer instances of “puddling” or “pooling” directly above the moisture-repellent fabric.

Fabric 3 appears to have provided better leakage protection than Fabric 4, perhaps due to the slightly higher weight of Fabric 3.

The results of the panty tests showed that the performance was dependent upon a variety of factors including the hand, weight, absorbency and perceived thickness of the gusset.

The best results were obtained using the panty construction in Example 8. The panty in Example 8 was constructed such that the brushed surface of each layer of Fabric D was facing away from the body and the sueded surface of the layer of Fabric 6 was also facing away from the body. In addition, the width between leg openings of the inner gusset layer D was narrower than that of the outer layer 6, and the layers were attached by sewing using a clean finish. As a result, the outer layer (6) rolled over into the gusset region, and the leg opening seam was disposed on the inside of the gusset region, spaced apart from the edge of the leg opening. Neither the inside or outside of the gusset region had any exposed seam threads. In certain preferred embodiments, the width of the lining 14 and the shell 12 between the leg openings is approximately equal, and the seam has exposed seam threads.

It has been found that the use of non-wicking thread and non-wicking or moisture-repellent elongate elastic members substantially improved the performance of the garments of the present disclosure. In certain preferred embodiments, non-wicking clear elastic tapes were used in the elasticized regions (Fulflex, Inc., Brattleboro, Vt.). In other embodiments, elastic tapes were treated to improve moisture-repellent characteristics. Suitable moisture-repellent compositions for treating elongate elastic members include NT-X620 (a proprietary perfluoroalkyl acrylic polymer emulsion)and NT-X628 (a proprietary complex polymer emulsion), both available from Nano-Tex, Oakland, Calif.

Several forms of non-wicking thread are known in the art, including polyester thread. In preferred embodiments of the present disclosure, the thread is a non-wicking, nylon thread. Non-wicking nylon thread previously only available for medium- and heavy-duty applications, has been developed at a size suitable for panty construction. In certain particularly preferred embodiments, the non-wicking nylon thread is ANEFIL® Nylon Dry Tex 21 STX (American & Mt. Holly, N.C.).

### Air Permeability Testing

The air permeability of several commercially available panties was measured and compared to the panties constructed as in working Example 8. The results are shown in Table D, below:

TABLE D
Air Permeability Test

| | | Air Permeability | Average |
|---|---|---|---|
| Panty Type | Test # | (ft³/in/ft²) | (ft³/in/ft²) |
| BFF Period Undies | 1 | 0.833 | — |
| VV Skivvys | 1 | 0.287 | — |
| Hanky Panky | 1 | 48.6 | 49 |
| Cotton Thong | 2 | 49.3 | |
| Natori Nylon Bikini | 1 | 64.6 | 63.5 |
| with Cotton Crotch | 2 | 62.4 | |
| Eulie 3-ply Fabric | 1 | 45.6 | 44.8 |
| (from Example 8) | 2 | 45.7 | |
| | 3 | 43.1 | |
| | 4 | 44.3 | |
| | 5 | 45.4 | |

In comparison to the Eulie panties, the BFF Period Undies and the VV Skivvies panties were relatively bulky, crinkly, did not move easily, and did not breathe. In contrast, the Eulie panties fit smoothly, moved easily, and breathed well, which is reflected in the air permeability values shown in Table D above. As shown the air permeability of the Eulie panties was comparable to a cotton thong, whereas the commercially available “period panties” from BFF and VV Skivvies had extremely low air permeability.

The moisture-absorbent lining of the Eulie panties of working Example 8 was compared to the inner cotton liner of the commercially available Knock Out™ panties, www.knockoutpanties.com. In this modified dip test, fabric samples 1.4375″ (36.5 mm) by 5.1875″ (131.7 mm) were used. The dry fabric samples were weighed, then fully submerged in water for 15 seconds, the removed from the water and held in air by the upper corners for 25 seconds to let excess water drain off. The results are presented in Table E, below. The moisture-absorbent lining, fabric D, of an embodiment of the present disclosure absorbed more water per gram of fabric than the cotton liner of the commercially available Knock Out panties. These results indicated that a lining of two layers of fabric D, such as the embodiment of Example 8, would have a total water absorbency of about 1054 g/m², consistent with the measurement reported in Table A.

TABLE E
Moisture Absorption

| | | | | Weight |
|---|---|---|---|---|
| | Dry Wt. | Saturated | Water Weight | absorbed per g |
| Fabric | (g) | wt. (g) | absorbed (g) | of fabric (g) |
| Fabric D (United | 0.6 | 3.0 | 2.4 | 3.9 |
| Knitting style | | | | |
| 65982, sample 1) | | | | |
| Fabric D (United | 0.7 | 3.2 | 2.6 | 3.8 |
| Knitting style | | | | |
| 65982, sample 2) | | | | |
| Knock Out | 1.2 | 4.4 | 3.2 | 2.8 |
| Cotton Liner | | | | |

While the disclosure has been described with reference to exemplary embodiments, it should be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended Claims.

What is claimed is:

1. A washable undergarment comprising:
at least one moisture-absorbent layer of fabric having a first body-contacting side and a second side, and at least one moisture-repellent layer of fabric having a first side disposed adjacent to the second side of the moisture-absorbent layer, and a second side; and
a leg cuff comprising a portion of the at least one moisture-repellent layer of fabric, a seam and an elongate elastic member.

2. The undergarment of claim 1 wherein the undergarment comprises a lining or a gusset having 1-4 moisture-absorbent layers and a shell having 1-2 moisture-repellent layers.

3. The undergarment of claim 1 wherein the seam comprises non-wicking thread

4. The undergarment of claim 3 wherein the thread is a nylon thread.

5. The undergarment of claim 1 wherein the elongate elastic member further comprises a moisture-repellent composition.

6. The undergarment of claim 1 wherein a single fabric layer weighs about 3.7 oz/yd² to about 7.2 oz/yd².

7. The undergarment of claim 1 wherein a single fabric layer weighs about 7 oz/yd² to about 14.6 oz/yd².

8. The undergarment of claim 2 wherein the combined fabric layers weigh about 9.5 oz/yd² to about 24.9 oz/yd².

9. The undergarment of claim 1 wherein a single fabric layer has a total water absorbency of about 347 to about 806 g/m².

10. The undergarment of claim 2 wherein the total absorbency of the combined fabric layers is about 806 g/m² to about 1178 g/m².

11. The undergarment of claim 2 wherein the combined fabric layers have an absorbent capacity of about 38 to about 50 ml of fluid.

12. The undergarment of claim 2 wherein the lining comprises a front edge, a first surface, a back edge and opposing side edges, and the undergarment has a front and a back, wherein the front edge of the lining is attached to the front of the undergarment at a front seam and the back edge of the lining is attached to the back of the undergarment at a back seam.

13. A garment portion comprising:
at least one moisture-absorbent layer having a first body-contacting side and a second side; and
at least one moisture-repellent layer having a first side disposed adjacent to the second side of the moisture-absorbent layer, and a second side, wherein the garment portion has a total water absorbency of about 347 to about 1054 g/m².

14. The garment portion of claim 13 wherein the garment portion is a gusset or a lining.

15. The garment portion of claim 13 comprising 1-4 moisture-absorbent layers and 1-2 moisture-repellent layers.

16. The garment portion of claim 15 wherein the total water absorbency of the combined fabric layers is about 806 g/m² to about 1178 g/m².

17. The garment portion of claim 13 further comprising non-wicking thread.

18. The garment portion of claim 15 wherein the combined fabric layers have an absorbent capacity of about 38 to about 50 ml of fluid.

19. The garment portion of claim 15 wherein the combined layers weigh about 9.5 oz/yd² to about 24.9 oz/yd².
